(19) United States
(12) Patent Application Publication
(10) Pub. No.: US 2009/0099428 A1
(43) Pub. Date: Apr. 16, 2009

(54) Method of blood oxygen saturation by ophthalmic measurement

(75) Inventors: Rejean J. Munger, Ottawa (CA); Orazio Berolo, Ottawa (CA)

Correspondence Address: FREEDMAN & ASSOCIATES, 117 CENTREPOINTE DRIVE, SUITE 350, NEPEAN, ONTARIO, K2G 5X3, CA

(73) Assignee: Evasight Instruments Inc., Ottawa (CA)

(21) Appl. No.: 12/285,334

(22) Filed: Oct. 2, 2008

Related U.S. Application Data
(60) Provisional application No. 60/960,540, filed on Oct. 3, 2007.

(51) Int. Cl. A61B 5/1455

(52) U.S. Cl. 600/318

(57) ABSTRACT

A method of performing a diagnostic characterization and assessment of a patients' eye is provided. The method comprising the extraction of measurement data relating to a predetermined portion of the eye, for example the fovea, at a plurality of wavelengths and fitting the extracted measurement data to a model of the eye, for example a linear adaptive model. The data subsequently analyzed to determine at least one of a common retinal behavior, a deviation from the common retinal behavior, and an oximetry value. In the case of the measurement data relating to the fovea of the eye then the oximetry value is indicative of the blood oxygen saturation of the choroid vasculature of the eye. Further analysis based upon storing the measurement data and/or analysis from each test allows for the changes within the patients eye to be established unlike prior art solutions, and identifying the changes with an element of the eye such as the retina, cornea, crystalline lens, etc.

### FIELD OF THE INVENTION

The invention relates to a method of measurement of blood oxygen saturation and more particularly a non-invasive test based upon ophthalmic measurements.

### BACKGROUND OF THE INVENTION

In medicine, oxygen saturation (S_{O2}) measures the percentage of hemoglobin binding sites in the bloodstream occupied by oxygen. Each hemoglobin molecules in blood has 4 sites for binding with oxygen or CO2. The binding of gas molecules occurs in the presence of a gas mixture as a function to the partial pressure of the gas in the mixture. At low partial pressures of oxygen, most hemoglobin is deoxygenated, meaning some of the oxygen binding sites are not occupied by oxygen and the blood carries less than its optimal amount of oxygen. In an oxygen rich environment (high partial pressure) blood gathers oxygen and each site of the hemoglobin molecule is fully bound to oxygen and it is said to be fully saturated. At around 90% (the value varies according to the clinical context) oxygen saturation increases according to an oxygen-haemoglobin dissociation curve and approaches 100% at partial oxygen pressures of >10 kPa. Oxygen saturation (S_{O2}) being specified in percent and determined from the ratio of occupied oxygen binding site to the maximum number of sites available. Since saturation is a direct indication of the oxygen partial pressure surrounding blood, it is an indicator of oxygen used by those cells consuming oxygen in that region and thus an indication of their metabolic activity and or the permeability of the blood vessel to allow the oxygen to reach the tissue.

Low arterial oxygen saturation being termed hypoxemia. This may be caused by various medical conditions including but not limited to low inspiration, low inspired fractional concentration of oxygen, alveolar hypoventilation, impairment of diffusion across blood-gas membrane, a shunt (intracardiac or intrapulmonary), and ventilation-perfusion inequality.

Worldwide health care costs have reached in excess of $4.5 trillion worldwide. A recurring theme in analysis of how to stem the growth of these health costs has focussed in many instances to improving the accuracy of medical tests, improving the accessibility of routine diagnostic testing to enhance early detection and treatment, and accordingly activities addressing non-invasive diagnostic techniques and their transferal to environments other than hospitals and specialist medical centres.

At present the most common means of estimating the oxygen saturation non-invasively is easily and painlessly with a clip that fits on the finger or earlobe. This clip, and the machine interfaced to it, exploits the varying optical absorption of blood with oxygenation. However, whilst non-invasive, quick and cheap these machines only provide a rough estimate as the measurement is affected by manner factors not calibrated into it such as the skin color of the individual, materials on the skins surface such as oils, moisture, nail polish, etc.

However, at present physicians seeking a reasonably accurate measure of oxygen saturation must currently resort to the arterial blood gas test. For this test, a small sample of blood must be drawn directly out of an artery. Most routine blood tests use blood that is drawn out of a vein, so this test is a little different. The artery that is sampled most often is the radial artery, and whilst the procedure takes only a few seconds the blood is typically not analyzed immediately but rather taken to a blood analysis laboratory. Whilst the blood is within a sealed syringe when taken to the blood gas analyzer care must be exercised to chill it in order to slow the metabolic processes that otherwise modify blood gas mixtures and result in an inaccuracy.

Accordingly currently there exists the need for a truly non-invasive measurement of blood oxygen. It would be further beneficial if the measurement was made by adapting an existing standard piece of medical test equipment, thereby reducing significantly the barrier to its adoption and proliferation. It would be similarly beneficial if the procedure provided additional medical information allowing trends to be obtained from multiple measurements over a period of time thereby enhancing the preventative aspects of such measurements as well as the immediate provision of a blood oxygen saturation.

It is the purpose of this invention to propose a method of measuring the oxygen saturation of the fovea.

### SUMMARY OF THE INVENTION

In accordance with the invention there is provided a method of diagnosis comprising:

(a) providing an optical source, the optical source capable of providing an illumination into a eye at at least one predetermined wavelength of a plurality of wavelengths, each predetermined wavelength having a predetermined spectral bandwidth;

(b) providing an optical detector, the optical detector capable of capturing an image of a predetermined portion of the eye when illuminated with the optical source;

(c) extracting at least one measurement data of a plurality of measurement data from the optical detector, the plurality of measurement data established according to a predetermined mask;

(d) applying the at least one measurement data to a model; and

(e) performing an analysis upon the at least one measurement data.

In accordance with another embodiment of the invention there is provided a method comprising:

(a) performing at least one set of optical measurements comprising a predetermined series of images of a predetermined portion of the eye of a patient, each of the predetermined series of images associated with one wavelength of a predetermined sequence of wavelengths;

(b) storing a predetermined portion of a current set of optical measurements within a digital memory, the stored current set of optical measurements associated with at least one of a time stamp and an identity of the patient.

In accordance with another embodiment of the invention there is provided a method comprising combining a model of a eye and at least one set of optical measurements of an eye of a patient to perform a diagnostic assessment of the patients eye, the at least one set of optical measurements comprising a predetermined series of images of a predetermined portion of the patients eye associated with a predetermined sequence of illuminating wavelengths.

In accordance with another embodiment of the invention there is provided a method comprising combining a model of a eye and at least one set of optical measurements of an eye of a patient to perform a diagnostic assessment of the patients eye, the at least one set of optical measurements comprising a predetermined series of images of a predetermined portion of the patients eye associated with a predetermined sequence of illuminating wavelengths.

In accordance with another embodiment of the invention there is provided a combining a model of a eye and at least one set of optical measurements of an eye of a patient in performing an assessment of the patients eye, the at least one set of optical measurements comprising a predetermined series of images of a predetermined portion of the patients eye associated with a predetermined sequence of illuminating wavelengths.

In accordance with another embodiment of the invention there is provided a method comprising

(a) testing a patient for a first optical characteristic, the testing performed using a first ophthalmic instrument on an eye of the patient;

(b) obtaining at least one set of optical measurements of an eye of the patient, the at least one set of optical measurements comprising a predetermined series of images of a predetermined portion of the patients eye associated with a predetermined sequence of illuminating wavelengths;

(c) subsequently processing the at least one set of optical measurements in dependence upon a least a model of the eye.

### BRIEF DESCRIPTION OF THE DRAWINGS

Exemplary embodiments of the invention will now be described in conjunction with the following drawings, in which:

FIG. 1 illustrates the optical absorption spectra for oxygenation and non-oxygenated hemoglobin.

FIG. 2 illustrates the complexity of ophthalmic measurements due to the multiple optically transparent and absorbing materials.

FIG. 3 illustrates the cell layers above the receptors and the receptors themselves within the human eye.

FIG. 4 illustrates the retinal vasculature around the fovea of the human eye.

FIG. 5 illustrates the density of receptor cones within the human eye.

FIG. 6 illustrates the target region of the human eye for the blood oxygenation measurements and the multi-sector target employed.

FIG. 7 illustrates a typical workflow for measurements and analysis.

### DETAILED DESCRIPTION OF EMBODIMENTS OF THE INVENTION

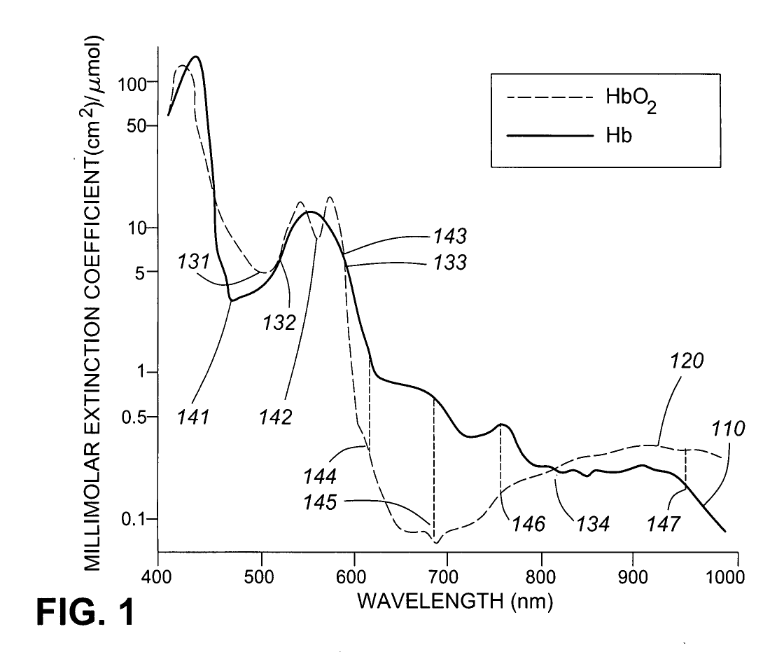

It is well known that hemoglobin has significantly different light absorption properties if it is unbound to oxygen (deoxyhemoglobin, Hb) or bound to oxygen (oxyhemoglobin, HbO₂), as illustrated in the spectra 110 and 120 respectively of FIG. 1. The light absorption properties of in-vivo blood is usually neither, it usually has a saturation less than 100% (except in the lung) but >65% in the vessels returning the blood from metabolically very active areas of the body. Furthermore, there are many other metabolic products in the blood that affect the absorption spectrum. However, by far the dominant absorption component is haemoglobin. Further the absorption of light of partially oxygenated hemoglobin is simply a weighted sum of the spectrum of Hb and HbO2,

A(λ)=a HB(λ)+b HbO2(λ), (1)

where a and b are two dimensionless coefficients. The oxygen saturation is simply S=b/(a+b)*100. If we were working with pure hemoglobin, this approach would work perfectly, and it is this factor that leads to the errors from the simple clip based optical techniques for measuring oxygen saturation. But real blood contains many species of hemoglobin, including but not limited to metahemoglobin, sulfur bound hemoglobin, and carbon dioxide bound hemoglobin, plus many other metabolites which can interfere with the accuracy of simple optical measurements.

However, detailed analysis of the Hb spectra 110 and HbO₂ spectra 120 in FIG. 1 shows that at a number of different wavelengths there are significant variations in the optical absorption for hemoglobin that differentiate oxyhemoglobin from deoxyhemoglobin. More specialist medical instruments, rather than the simple clip use light to detect oxygen saturation in fingers or in blood samples and simplify the saturation measurement by not taking a complete spectrum but by looking at between 2 to 6 different wavelengths and comparing their relative amplitudes. The expectation from this is that some of these ratios remove the impact of confounding factors in the overall measurement. For example, if the instrument uses the isobestic points where the absorption is of equal value in Hb spectra 110 and HbO₂ spectra 120 such as λ=506 nm being isobestic point 1 131, λ=522 mm being isobest point 2 132, λ=586 nm as isobest 3 133 and λ=815 nm being isobest point 4 134 relative to other points of interest then in principle the impact of these confounding factors that affect most of the absorption should be removed from the measurement. Such other points of interest being wavelengths A 141 through wavelength G 147 for example. These approaches have limited success are removing confounding factors introduced by individual variations, environmental factors (deposits on skin, sweat, etc.) when doing an optical measurement through human tissues. Furthermore, the usually low transparency of most of human tissues means that the blood related signal recovered from the tissue is usually a very small portion of the optical signal, reducing the quality and reliability of the oximetry measure (oximetry being the determination of oxygen-hemoglobin saturation of blood).

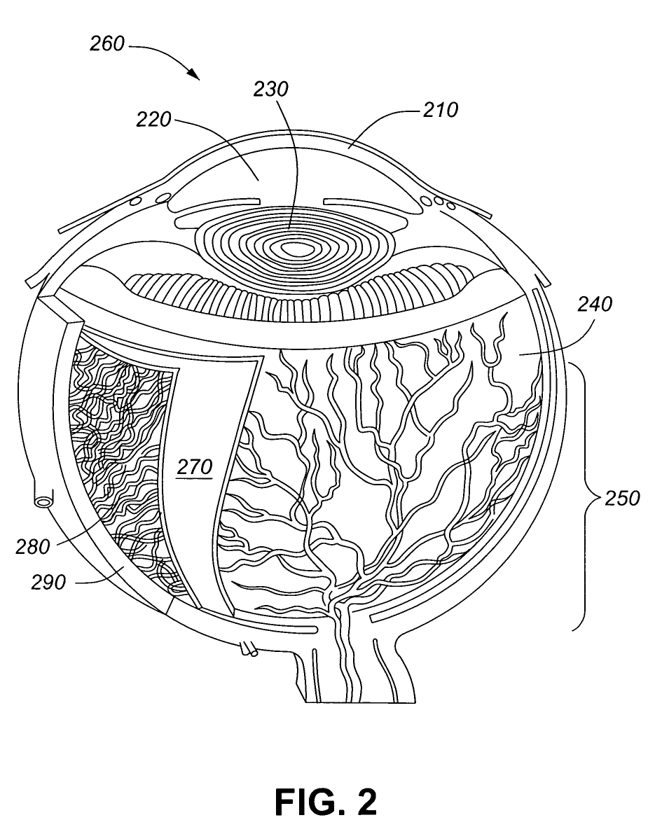

The eye with its high transparency and surface vasculature offers an opportunity to perform oximetry measurements optically that is less dependent than on tissue variability as described above and completely non invasive. However, the optics of the eye are complex and this must be taken into account as illustrated by the schematic 200 of the human eye in FIG. 2. The light entering the eye must go through the cornea 210, the aqueous humour 220, the crystalline lens 230 and the vitreous humour 240 before it interacts with the vasculature posterior segment 250 of the human eye 260. Each of these components, cornea 210, the aqueous humour 220, the crystalline lens 230 and the vitreous humour 240, having its own light absorption properties. Furthermore, the eye has two vascular systems, one of which is readily visible in photography and is composed of vessels at the surface of the retina 270, namely those layers at the back of the eye responsible for vision, with capillaries branching into the anterior part of the retina. The second is a denser vasculature, the choroids 280, that sits behind the retina just before the sclera 290, this being the hard tissue enveloping the whole human eye 260, which feeds the deeper tissues of the retina 270. Almost all oximetry of the eye has been done using surface vessels sitting on the surface of the retina because the light absorption is dominated by the optical properties of the blood in the vessels.

Here again, prior art approaches have typically used between 2 and 6 different wavelengths to obtain oxygen saturation, without taking into account the optical properties of the whole eye. In some research reports these has been reported the fitting of the whole visible spectrum returned from such blood vessels to extract the oxyhemoglobin and deoxyhemoglobin absorption properties and thereby obtain oxygen saturation with seemingly good success. Such techniques being slow unless a single point only is assessed and performed in laboratories and not compatible with asking a patient to sit still in front of a slowly varying optical source so that the measurements may be taken on a group of point in a consistent part of the patients' eye to ensure diagnostic value.

However, the inventors have found that the discrete wavelength approach may be improved by taking into account the optical properties of the retina and the other optical components of the eye to account for their impact on the measured spectrum rather than either ignoring them or relying upon isobestic measurements to remove them.

As such an embodiment of the invention is based a physically realistic model of the optical absorption of the eye such as but not limited to the following linear model

A(λ)=a HB(λ)+b HbO2(λ)+c P_{L}(λ)+d P_{M}(λ)+e P_{S}(λ)+f Co(λ)+ (2)

g CL(λ)+L₁(λ)+L₂(λ)+ . . . +Lₙ(λ)

where absorption is measured in absorbance units, being the logarithm of absorption. Accordingly within Equation (2) P (L,M,S) refer to the three types of cones within the human eye, Co is the cornea, CL the crystalline lens, and the Ln terms represent any number of layers in the retina such as the retinal pigmented epithelium, Bruch's membrane, melanin, the sclera and others as depicted within FIG. 2 as appropriate. The CL term typically being two terms one presenting the response of the lens in the elderly and another representing the response of the lens in the young. However, such terms are not represented in the embodiments of the invention for reasons of simplicity. Whilst the embodiments of the invention discussed employ a linear adaptive model, other models of the eye can also be employed without departing from the scope of the invention.

Because the retinal layers are below the retinal blood vessels, their contributions are small and it is possible to extract oxygen saturation with a good level of quality. However, two issues exist. Firstly, photoreceptors are activated by light so that as we shine light on the retina we activate them and thus we are looking at an active system. As a result prior art solutions that employ pulsed high intensity light oximetry are not as reliable as those measurement techniques that employ constant lower light level.

Secondly, these blood vessels within the human eye above the retina are not indicative of the blood oxygen saturation within the artery-vein system of the patient. To obtain this the measurements should be made using the choroidal vessels within the human eye. However, for most of the human eye it is very difficult to measure choroidal oxygen saturation as these retinal surface signals overwhelm the choroidal signal. This arises as the choroid vasculature is deep in the retina behind the pigmented epithelium which can absorb a large amount of light, as do other retinal layers. Accordingly, the optical signal reaching the choroid vasculature is normally very highly attenuated and structured with many optical absorption features from these preceding layers.

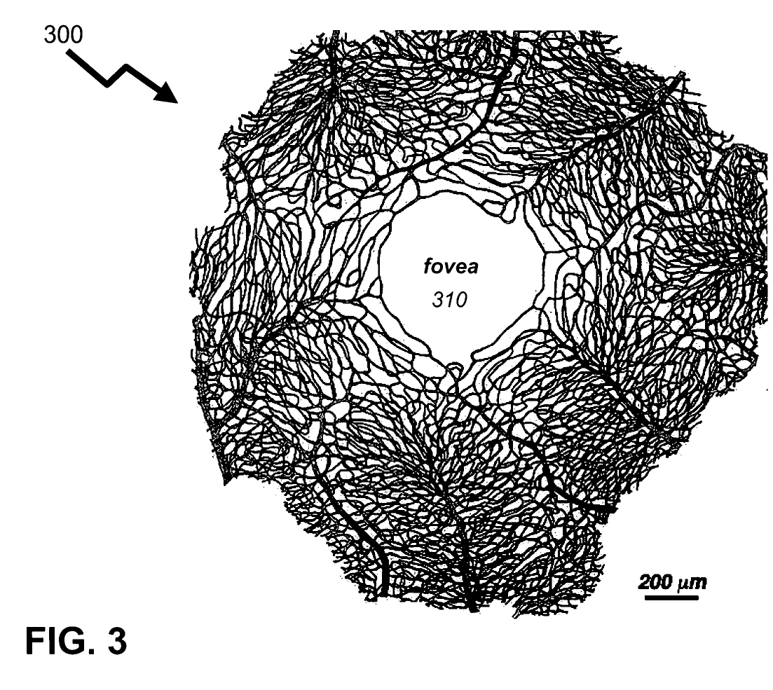

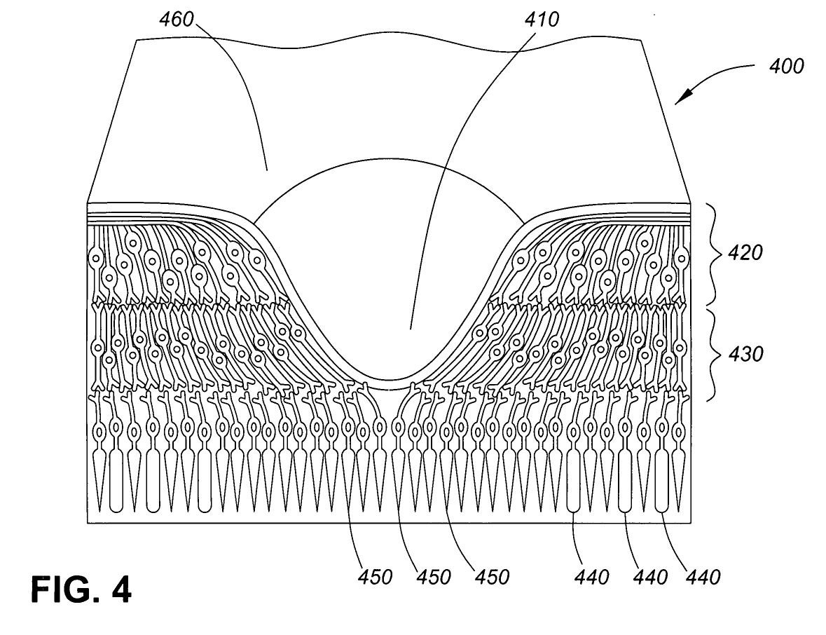

However, as illustrated in FIG. 3 there is a region of the back of the human eye 300 called the fovea 310. This region of the eye has no surface vasculature 320. Further, as shown in the schematic cross-section 400 of FIG. 4 it is significantly thinner than any other part of the retina. Further, as this region of the eye provides the highest quality vision then damage to or loss of this region through disease has the greatest impact on the patients' quality of life. As a result monitoring this region of the eye, the fovea 410, has enhanced benefits. Further the oxygen related optical signal from the fovea 410 is completely determined by the choroidal vasculature as it is the only oxygen source for the fovea 410. Within the prior art measurements within this region of the human eye have never been done with any reliability. As shown within FIG. 4 the upper ganglion cell layer 420 and bipolar cell layer 430 reduce towards the center of the fovea 410. Similarly, the population of photoreceptors adjusts from one of rods 440 and cones 450 to solely cones 450.

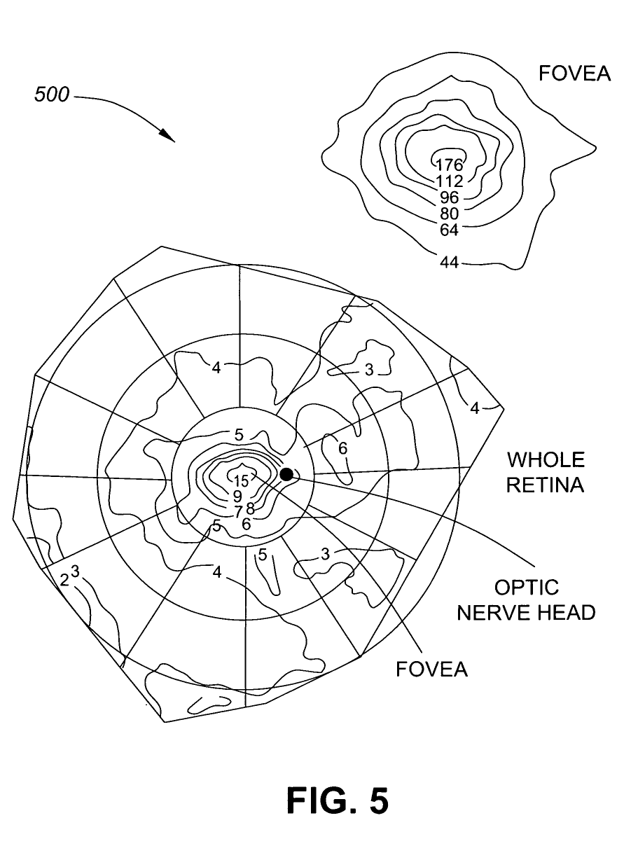

However, simply performing multi-wavelength measurements within the fovea 410, even using the linear additive model of Equation (2) does not provide the necessary data for an accurate reading of the blood oxygen saturation of the choroidal vasculature. This arises as the retinal layers of the retina in the fovea vary radially in respect of the density of the photoreceptors as evident from the isodensity map 500 of FIG. 5. As shown the density of cones, from Curcio et al, within the human retina varies from a few thousand across the majority of the retina but rapidly rises at the edge of the fovea 410 to approximately 40,000 to in excess of 170,000 at the centre. As such these densities reach a factor of approximately 30,000 times those over the bulk of the retina and vary approximately four fold across the fovea 410 itself.

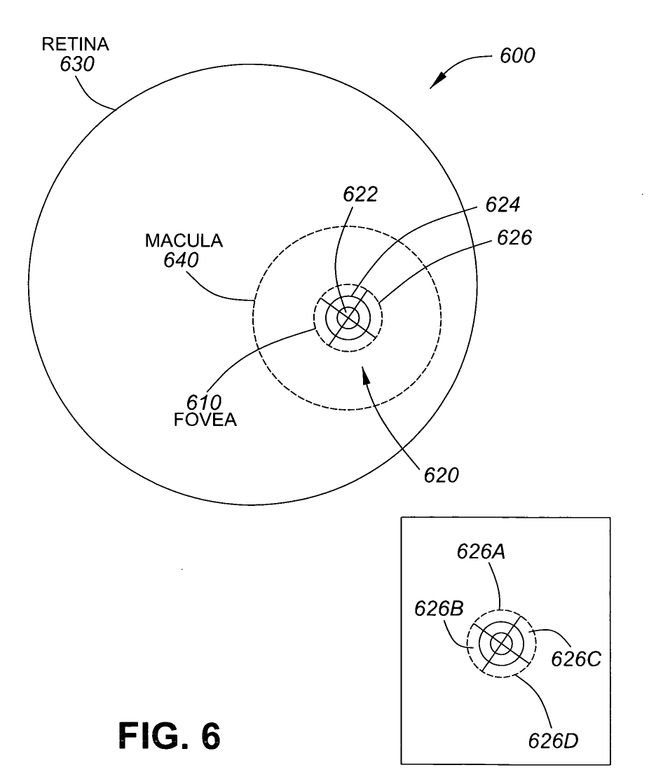

As is evident from the isodensity map 500 the cone density varies with an approximately circular symmetry. Accordingly, the absorption and optical scattering properties of all other retinal tissue within a healthy eye should also follow this same symmetry. Hence, as depicted in the target analysis image 600 of FIG. 6 the fovea 610 is broken down into a target 620 comprising three rings, inner ring 622, middle ring 624, and outer ring 626. Each of the rings then broken into quadrants, such as exemplified by outer ring 626 which is formed from quadrants 626A through 626D. In this manner the radial variations of the fovea 610 may be utilized in the measurement data to ensure that the measurements are firstly made within the fovea 610, as no radial variation would exist in the bulk of the retina, and secondly correctly centered on the fovea 610.

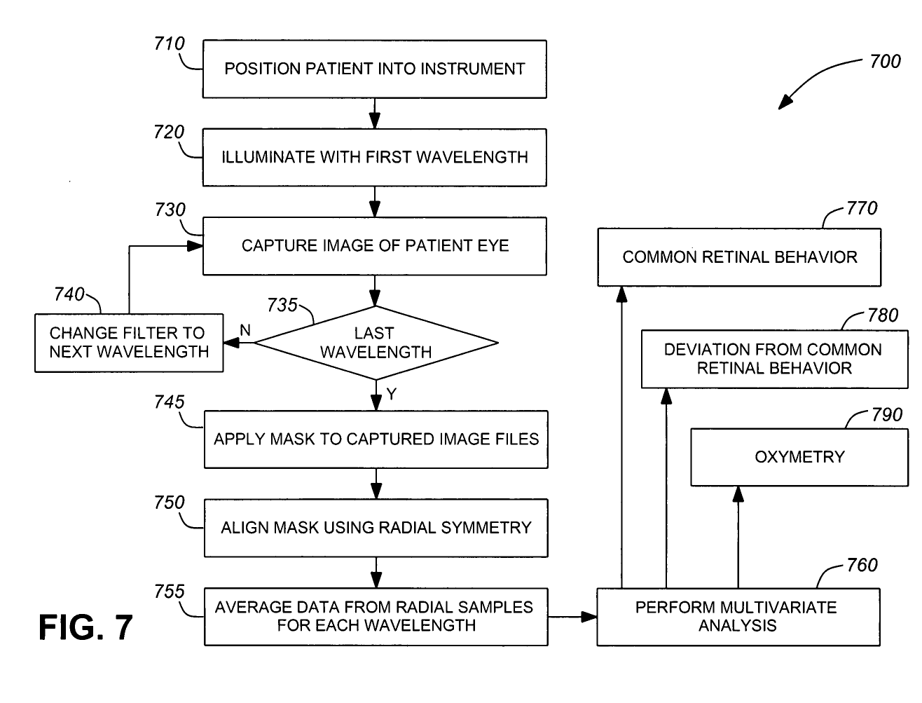

As noted supra it is not possible to obtain a full spectrum at each point in the fovea in a reasonable time for a clinical application. Eye move, patient fatigue, and metabolic status changes would occur over a few seconds compared with the data acquisition taking several minutes. Hence, as presented in FIG. 7, an embodiment of the measurement workflow 700 is provided wherein the spectrum is estimated from a few wavelength points obtained from digitized pictures of the retina taken with different band limited filters, as shown in respect of steps 710 through to 735 inclusively.

An adaptive model of the absorption for the human retina, such as for example Equation (2), cannot be solved for the many terms to extract the desired result, namely the oxygen saturation. As such, with insufficient data the system is undetermined, an issue that is further compounded by the fact that the choroid vasculature's signals are not the dominant factor as for the case of surface retinal vessels thereby yielding a low signal to noise ratio. Hence, as shown in steps 745 through to 750 the analysis is performed on the basis that data from regions of the retina equidistant from the fovea 610, where all the retinal layers are similar, would have the same optical properties. In this manner all the layers are treated as a single system with complex behavior that is a sum of its components, the two other components therefore being the Hb and HbO2 components of the choroidal vasculature absorption characteristics.

Having centralized the data according to steps 745 through 750 the average responses of the similar retinal points are fit to the model in step 760, the averages still being limited to the few data points from each filtered light picture. As a result in step 760 a multivariate analysis is performed to extract the most probable common behavior of all the retinal points which are expected to be similar. From this analysis three sets of data are obtained, the common retinal behavior for a set of retinal points 770, the deviation from common behaviors 780, and oximetry from each retinal point 790.

Each of these data sets is then employed in an analysis for the patient, providing an indication of abnormal behavior and thereby affording an early detection of problems in the retina. In some instances like oximetry in step 790 the data would be directly correlated, whilst for others it would be compared to “normal” values for the identification of abnormal properties.

Further, for step 760 the decomposition could be further extended using the model of the absorption of the eye, such as for example Equation (2), to obtain the optical properties of the cornea, lens, etc and subtracting them from the fovea data. This data providing in the first ophthalmic examination a baseline data set allowing with subsequent repeat examinations this data to be subjected to additional analysis such as trend analysis or outlier analysis. In this manner physicians are able to determine degradations in the crystalline lens, disease on the fovea, etc.

Whilst the embodiment presented supra in respect of FIG. 6 presents one possible measurement pattern, other patterns may be employed, some of these providing for either an increase or a decrease in the size of the expected common behavior of the measurements and response of the human eye.

Additionally, whilst typically across the macular 620 and remainder of the retina 630, external to the fovea 610, the density of photoreceptors is relatively constant, compared to the steep gradient of the fovea 610, the approach would provide a comparable ability to determine diseased regions or establish abnormal responses relative to the accumulated data of normal measurements. Whilst the error in such measurements may be increased the reduced optical acuity in these regions counterbalances this.

Whilst the embodiments of the invention discussed supra are in respect of the human eye the method and embodiments are applicable to other eyes without departing from the scope of the invention.

Numerous other embodiments may be envisaged without departing from the spirit or scope of the invention.

What is claimed is:

1. A method of diagnosis comprising:
(a) providing an optical source, the optical source capable of providing an illumination into a eye at at least one predetermined wavelength of a plurality of wavelengths, each predetermined wavelength having a predetermined spectral bandwidth;
(b) providing an optical detector, the optical detector capable of capturing an image of a predetermined portion of the eye when illuminated with the optical source;
(c) extracting at least one measurement data of a plurality of measurement data from the optical detector, the plurality of measurement data established according to a predetermined mask;
(d) applying the at least one measurement data to a model; and
(e) performing an analysis upon the at least one measurement data.

2. A method according to claim 1 wherein,
capturing an image of the predetermined portion of the eye comprises capturing an image of at least one of a predetermined portion of a fovea region of the eye, a predetermined portion of a macular region of the eye; and a predetermined portion of the retina.

3. A method according to claim 1 wherein,
step (c) comprises taking the at least one measurement data at a predetermined time, the predetermined time being at least one of relative to a selected point in a cardiac cycle, after a predetermined delay from illuminating the eye in step (a), and at a predetermined point in a time sensitive clinical measurement.

4. A method according to claim 1 wherein,
step (e) comprises determining at least one of a common retinal behavior, a deviation from the common retinal behavior, and an oximetry value, the oximetry value being determined in dependence upon at least a hemoglobin contribution and an oxyhemoglobin contribution to the at least one measurement data.

5. A method according to claim 1 wherein,
step (d) further comprises re-aligning the at least one measurement data of the plurality of measurement data according an aspect of symmetry of the plurality of measurement data.

6. A method according to claim 1 wherein,
providing a predetermined mask comprises providing at least one set of measurement coordinates, the set of measurement coordinates distributed radially at a predetermined separation from a nominal centre of the mask.

7. A method according to claim 1 further comprising:
(f) storing within a digital memory at least one of the at least one measurement data of a plurality of measurement data and the outcome of the analysis of step (e), the stored data associated with at least one of a time value and a client identity, the time value relating to the at least one of a time and a date of the at least one of one measurement data and the analysis.

8. A method according to claim 1 wherein,
step (e) comprises establishing at least one of a radial asymmetry in the at least one measurement data and establishing an optical property of the eye, the optical property relating to an element of the eye selected from a group comprising cornea, crystalline lens, fovea, macular, retina, choroid vasculature, retinal vasculature, aqueous humour, and vitreous humor.

9. A method according to claim 1 wherein,
the method of diagnosis according to claim 1 is performed at least one of together with another ophthalmic measurement without the patient moving, using an ophthalmic instrument providing at least another ophthalmic measurement, and a dedicated instrument.

10. A method according to claim 1 wherein,
applying the measurement data to the model comprises applying the data to at least one of an adaptive model of the eye and a linear adaptive model of the eye.

11. A method comprising:
(a) performing at least one set of optical measurements comprising a predetermined series of images of a predetermined portion of the eye of a patient, each of the predetermined series of images associated with one wavelength of a predetermined sequence of wavelengths;
(b) storing a predetermined portion of a current set of optical measurements within a digital memory, the stored current set of optical measurements associated with at least one of a time stamp and an identity of the patient.

12. A method according to claim 11 wherein,
storing a predetermined portion of the set of optical measurements comprises storing data relating to a predetermined series of measurement points, the series of measurement points established in dependence of a predetermined mask having an aspect of symmetry.

13. A method according to claim 12 wherein,
the series of measurement points are established after performing an initial step of determining a centre to which the predetermined mask is applied.

14. A method according to claim 11 further comprising:
(c) providing the predetermined portion of the current set of optical measurements to a model; and
(d) determining at least one of a common retinal behavior, a deviation from the common retinal behavior, and an oximetry value, the oximetry value being determined in dependence upon at least a hemoglobin contribution and an oxyhemoglobin contribution to the at least one measurement data.

15. A method according to claim 11 wherein,
the predetermined portion of the current set of optical measurements comprises that data relating to at least one of a predetermined portion of a fovea region of the eye, a predetermined portion of a macular region of the eye; and a predetermined portion of the retina.

16. A method according to claim 11 further comprising,
(c) extracting at least one previous set of optical measurements;
(d) performing an analysis of the at least one previous set and the current set of optical measurements;
(e) determining whether a change has occurred in the optical properties of the eye of the patient between the at least one previous set and the current set of optical measurements.

17. A method according to claim 16 wherein,
determining whether a change has occurred comprises performing at least one of a trend analysis between the at least one previous set and current set of optical measurements, comparing a difference between the at least one previous set and current set of optical measurements with a distribution of expected differences, comparing at least the current set of optical measurements with a distribution of optical measurements.

18. A method according to claim 17 wherein,
the at least one of the distribution of expected differences and distribution of optical measurements is established from an analysis of a plurality of eyes representing an approximately typical cross-section of a population of humans.

19. A method according to claim 17 wherein,
the at least one of the distribution of expected differences and distribution of optical measurements is established by filtering at least a database of optical measurements, the filtering performed in dependence upon at least a characteristic of the patient.

20. A method according to claim 19 wherein,
the at least a characteristic is selected from the group comprising sex, age, eye color, ethnicity, patients with cataracts, and patients with posterior vitreous detachment.

21. A method comprising:
combining a model of a eye and at least one set of optical measurements of an eye of a patient to perform a diagnostic assessment of the patients eye, the at least one set of optical measurements comprising a predetermined series of images of a predetermined portion of the patients eye associated with a predetermined sequence of illuminating wavelengths.

22. A method according to claim 21 wherein,
performing a diagnostic measurement comprises determining at least one of a blood oxygen measurement, a degradation within an element of the patients' eye and a disease associated with an element of the patients' eye.

23. A method comprising:
combining a model of a eye and at least one set of optical measurements of an eye of a patient in performing an assessment of the patients eye, the at least one set of optical measurements comprising a predetermined series of images of a predetermined portion of the patients eye associated with a predetermined sequence of illuminating wavelengths.

24. A method according to claim 23 wherein,
performing the assessment of the patients eye comprises assessing at least one aspect of the patients eye, the at least one aspect selected from the group comprising cornea, crystalline lens, fovea, macular, retina, choroid vasculature, retinal vasculature, iris, aqueous humour, and vitreous humor.

25. A method comprising:
(a) testing a patient for a first optical characteristic, the testing performed using a first ophthalmic instrument on an eye of the patient;
(b) obtaining at least one set of optical measurements of an eye of the patient, the at least one set of optical measurements comprising a predetermined series of images of a predetermined portion of the patients eye associated with a predetermined sequence of illuminating wavelengths;
(c) subsequently processing the at least one set of optical measurements in dependence upon a least a model of the eye.

26. A method according to claim 25 wherein,
step (b) is performed at least one simultaneously with step (a) and at least one of without the patient moving from the first ophthalmic instrument.
